(19) United States
(12) Patent Application Publication
(10) Pub. No.: US 2006/0253202 A1
(43) Pub. Date: Nov. 9, 2006

(54) Vertebral disc implant in fiber form

(76) Inventor: Eugene G. Lipov, Chicago, IL (US)

Correspondence Address: BRIAN J. LUM;ICE MILLER LLP, SUITE 3500, 200 WEST MADISON STREET, CHICAGO, IL, 60606, US

(21) Appl. No.: 11/374,494

(22) Filed: Mar. 13, 2006

Related U.S. Application Data
(60) Provisional application No. 60/677,966, filed on May 5, 2005.

(51) Int. Cl. A61F 2/44

(52) U.S. Cl. 623/17.16

(57) ABSTRACT

The present invention is directed to filamentous fibers and methods of use for treating a damaged disc, illustratively for treatment of a degenerative or injured vertebral disc. The filamentous fiber may be dehydrated such that filamentous fiber into the damaged disc such that the filamentous fiber absorbs fluid, expands, and provides cushioning to the damaged disc. In illustrative methods, the filamentous fiber is inserted into the disc via a cannulating needle.

This application claims priority to U.S. Provisional Application No. 60/677,966, filed May 5, 2005, herein incorporated by reference.

### BACKGROUND OF THE INVENTION

The present invention is directed to a thread-like implant and methods of using the implant to treat vertebral disc abnormalities.

The spinal disc is a cartilaginous spinal structure that allows the bending and rotation of the spine. The annulus of the disc is a tough outer fibrous ring that binds together the adjacent vertebrae. Inside the annulus there is a relatively liquid core, the nucleus. A healthy nucleus naturally has a high water content which aids in the load bearing and cushioning properties of the spinal disc.

Damage to the spinal disc can lead to dysfunction of the spine, serious pain, and often to long-term disability of a patient. A typical disc problem involves bulging or herniation of the spinal disc, wherein the nucleus of the spinal disc becomes extruded, which can then cause compression of an adjacent nerve and resulting inflammatory reaction. Herniation may occur due to trauma or to disc degeneration.

Age-related disc degeneration is progressive, leading to diminished nuclear water content and increased collagen content. These physiological changes in turn often lead to minor tears, which may lead to herniation. The physiological changes can also lead to decreased disc height, which can affect load-bearing, leading to pain even in the absence of herniation.

For herniated discs, it is common to perform a disc decompression, such as a laminectomy in which all or part of the disc nucleus is removed. The laminectomy results in decreased pressure on the nerve and often leads to significant pain relief. Typical surgical treatments for herniated or degenerative discs often include immobilizing the joint by fusing the neighboring vertebrae using various instruments and techniques, including bone grafts and rigid spinal cages. Such spinal fusion is invasive and results in some loss of flexibility in the spine. Artificial discs are known. However, they are also quite invasive, with mixed levels of success of maintaining flexibility with decreased pain.

### SUMMARY OF THE INVENTION

The present invention is directed to a minimally invasive treatment for disc degeneration or other disc abnormalities. Thread-like implants in the form of fibers with potential for absorption of fluid are provided through a cannulating needle to the degenerated disc. Illustratively, the fibers are made of hydrogel, collagen, or other suitable materials. Once provided to the disc space, the fibers expand by absorbing fluid naturally found in the intravertebral disc space. The expanded fibers provide cushioning, thereby relieving pain and retarding further disc deterioration.

Thus, in one aspect of the invention, an implant is provided for use in treating a damaged disc, the implant comprising a filamentous fiber having potential for absorbing fluid and packaged for insertion into a disc. The dehydrated filamentous fiber may be made of hydrogel, collagen, or other suitable materials.

In another aspect of the invention, the implant is packaged as a kit. In illustrative embodiments, the filamentous fiber is packaged on a spool or multiple filamentous fibers may be provided. The kit may include a delivery device, illustratively including a cannulating needle, optionally wherein the filamentous fiber is provided preloaded on a delivery unit configured for insertion through the cannulating needle.

In yet another aspect of the invention, methods are provided for treating a damaged disc, illustratively a vertebral disc, comprising the steps of providing a filamentous fiber configured to absorb fluid, and inserting the filamentous fiber into the damaged disc such that the filamentous fiber absorbs fluid, expands, and provides cushioning to the damaged disc. In illustrative methods the filamentous fiber is inserted into the disc through a cannulating needle. Optionally, the filamentous fiber may be chilled or frozen prior to insertion. The colder fiber would have more rigidity for improved ease of insertion. Once warmed to body temperature, the filamentous fiber would regain flexibility. Illustratively, the filamentous fiber also may be labeled, illustratively with a radioactive label or with a titanium fiber, and insertion and placement may be monitored radiographically. These methods may be performed in combination with disc nucleoplasty or other spinal disc treatments.

Additional features of the present invention will become apparent to those skilled in the art upon consideration of the following detailed description of preferred embodiments exemplifying the best mode of carrying out the invention as presently perceived.

### BRIEF DESCRIPTION OF THE DRAWINGS

FIG. 1 is a side view of a portion of a spine.

FIG. 2 is a cross-sectional view of a herniated disc of the spine of FIG. 1.

FIG. 3 is a cross-sectional view of a normal disc of the spine of FIG. 1.

FIG. 4 is a cross-sectional view of a degenerative disc of the spine of FIG. 1.

FIG. 5 is a cross-sectional view of the degenerative disc of FIG. 4, showing the insertion of an implant of one embodiment of the present invention.

FIG. 6 is a cross-sectional view of the degenerative disc of FIG. 4, showing multiple implants of one embodiment of the present invention.

### DETAILED DESCRIPTION

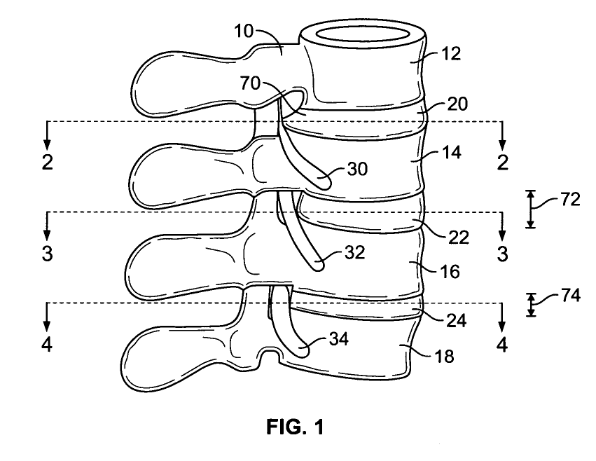

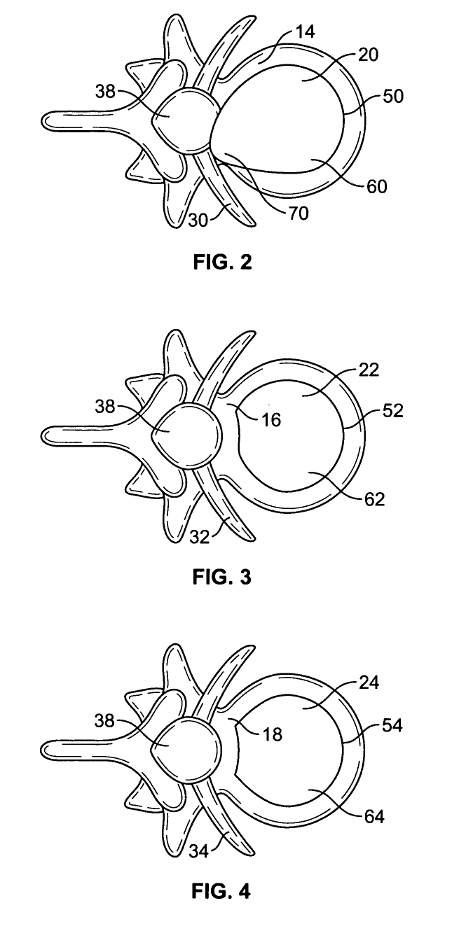

FIG. 1 shows a portion of a spinal column 10, showing four vertebrae 12, 14, 16, and 18. Spinal column 10 has a normal disc 22, best seen in FIG. 3. Normal disc 22 has an annulus 52 surrounding a normal nucleus 62. Normal disc 22 has a normal disc height 72 and normal disc 22 does not impinge upon the nerve 32.

Spinal column 10 also has a herniated disc 20. As best seen in FIG. 2, herniated disc 20 has an annulus 50 surrounding nucleus 60. However, nucleus 60 is bulging out at herniation 70, placing pressure on nerve 30. Herniated discs such as herniated disc 20 are often treated by removal of some or all of the nucleus 60. However, removal of some of the nuclear material can decrease disc height and decrease cushioning and load bearing properties of the disc. Accordingly, laminectomies are often performed along with spinal fusion.

FIG. 1 also shows a degenerative disc 24, also shown in FIG. 4. Degenerative disc 24 has an annulus 54 surrounding a degenerative nucleus 64. As best seen in FIG. 1, degenerative disc 24 also has reduced disc height 74, although disc degeneration does not always affect disc height, particularly in the earlier stages of degeneration. Where disc height is not yet affected, degeneration may be confirmed illustratively by discography and/or MRI.

Hydrogels have the ability to absorb fluid and expand. Various hydrogels are known, both for soft tissue repair and for various other medical uses. Ultrahigh molecular weight polyethylene (UHMWPE) hydrogels have been used for prosthetic spinal discs, for example the PDN Prosthetic Disc Nucleus (Raymedica, Inc., Bloomington, Minn.). Polyvinyl alcohol materials have also been used, as in the Aquarelle Hydrogel Nucleus (Stryker Howmedica Osteonic, Kalamazoo, Mich.). Hydrogels of other materials, such as silicone, polyacrylamide, and various other compositions are known. Many hydrogels can be made into a filamentous fiber that can be dehydrated, illustratively by lyophilization. The dehydrated fiber can absorb fluid and expand to a substantially larger size, providing cushioning within a disc space and helping to prevent further disc degeneration. Illustratively, the fiber may undergo a 10% to 1000% increase in volume, more illustratively a 20% to 50% increase in volume. However, it is understood that the precise amount of expansion will depend on the particular materials used to make the fibers. Accordingly, hydrogel fibers can be used for the treatment of degenerative discs.

In addition to the above hydrogels, fibers made of other materials are useful in the methods of the present invention. The fibers may be made of collagen, a material found in normal disc tissue, some of which may be lost during the degenerative process. Various extracellular materials are available, non-limiting examples including small intestine submucosa, stomach, bladder, alimentary, respiratory, or genital submucosa, or liver basement membrane. These extracellular materials can be formed into fibers in a variety of ways, including by extracting collagen and forming the collagen into fibers, by comminuting the extracellular material into long ribbon-like pieces and forming the comminuted extracellular material into filamentous fibers, and in various other ways. As with the hydrogel fibers, these fibers can be dehydrated, illustratively by lyophilization. These extracellular materials are known to have tissue regenerative properties and may be useful for regenerating some of the damaged tissue.

The fluid naturally found in the nucleus is acidic, and the acidic nature of this fluid adds to the pain experienced by the patient as this fluid escapes from the nucleus in herniated or degenerative discs. Thus, in one illustrative embodiment, the fibers used are basic. The basicity of such fibers would aid in neutralize the nuclear fluid. In one embodiment, the basic fibers would react with the nuclear fluid and act as a seal to seal the leaks in the annulus surrounding the nucleus. Various other substances that are known for their membrane-sealing potential may be used as well. Illustratively, polyalkylene glycols such as polyethylene glycol are known for their properties to aid in sealing natural membranes. These polymers may be used as part of the fibers to impart this function to the implant. The polyalkylene polymers may be provided along with the fiber material, or may be cross-linked to or woven into the fiber structure. Thus, in addition to cushioning, the fibers of the present invention may be used to decrease pain by neutralizing nuclear fluid and/or by aiding in sealing the leaks in the nuclear membrane.

The filamentous fibers of the present invention may be provided in any length suitable for placement in the intravertebral space, illustratively 1 mm, 2 mm, 3 mm, 4 mm, 5 mm, 6 mm, 7 mm, 8 mm, 9 mm, 10 mm, 12 mm, 15 mm, 20 mm, 30 mm, 40 mm, or 50 mm, or any length in between these lengths. Alternatively, long lengths, illustratively provided on spools or in other configurations, may be provided, and the surgeon can then cut the filamentous fiber to length before or during surgery. The spool may be a suitable supply of the filamentous fiber material for multiple patients. The filamentous fibers may be of any width suitable for implantation through a cannulating needle or other fluoroscopic technique. Illustrative widths are from 10μ to 5 mm, and more illustratively 0.1 mm to 2 mm. The filamentous fiber may be provided partially or fully pre-loaded into a cannulating needle, for ease of insertion, or may be provided as a kit wherein various pieces of the fiber are packaged together with or without the cannulating needle.

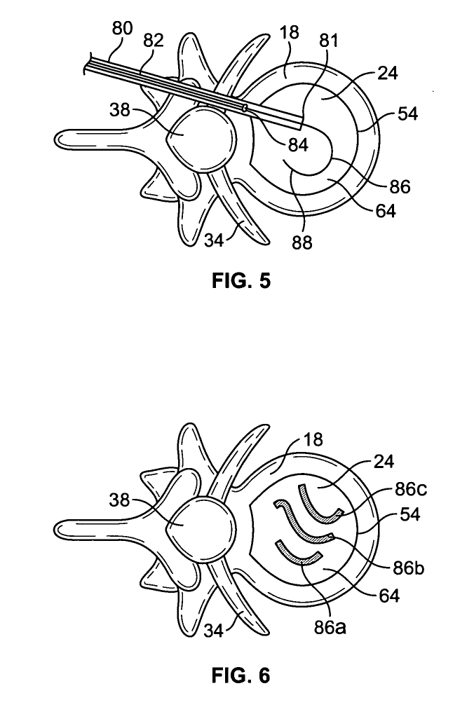

As shown in FIG. 5, a filamentous fiber 86 according to the present invention is inserted via a delivery unit, illustratively including a cannulating needle, into the intravertebral disc space occupied by degenerative disc 24. In the illustrated embodiment, filamentous fiber 86 is connected to a delivery unit 82. The surgeon can move delivery unit 82 toward and away from a tip 81 of cannulating needle 80. Once the filamentous fiber 86 is properly placed, rotation of the delivery unit 82 causes the filamentous fiber 86 to disengage from the delivery unit 82 at a connection point 84. Connection point 84 can be threaded, frangible, or any other configuration to provide for proper disengagement. Alternatively, the delivery unit can be a metal inner core that is not physically connected to the filamentous fiber inserts the filamentous fiber by pushing the filamentous fiber in an action similar to that of a plunger. Other configurations for the delivery unit are within the scope of this invention.

As shown in FIG. 5, fiber 86 is a fairly long fiber and extends most of the way across degenerative disc 24. The fiber may be provided in a fixed length, or may be cut to size either prior to insertion, or during or subsequent to insertion. In FIG. 5, only a single fiber is used. In another embodiment, FIG. 6 shows three shorter fibers 86a, 86b, and 8c already inserted adjacent degenerative disc 24. Any number and length of fibers may be used, as required by the particularities of the degenerative disc and judgment of the surgeon. Optionally, the fiber may be made rigid, illustratively by freezing, for ease of insertion.

In one embodiment, the fiber 86 is labeled, illustratively with a radioactive label or a small piece of titanium. The fiber may be labeled throughout its length or only partially labeled, illustratively at tip 88. When a labeled fiber 86 is used, insertion and placement of the fiber 86 may be assisted by radiography. Other labels, illustratively fluorescent dyes, may be used depending on the detection method.

Subsequent to insertion through this minimally invasive procedure with a cannulating needle, the fiber absorbs fluids naturally located in the intravertebral disc space. The fibers 86a, 86b, and 86c of FIG. 6 have already begun to absorb the fluids and have begun to expand. However, because degenerative discs often have a decreased water content, in some cases it may be desirable to inject a fluid into the disc space at the time the fibers are inserted. The fluid may be water, saline, or other biologically compatible fluid, and may contain one or more components found in the natural disc space, such as hyaluronic acid, glycosaminoglycans, collagens, growth factors, cartilage cells, or even cartilage progenitor cells. Other components may be injected as well. Such components as hyaluronic acid, glycosaminoglycans, and collagens assist the fibers in serving a biomechanical function, in lubricating the joint and maintaining the cartilage, while components such as growth factors and cells may provide a regenerative function. Thus, in one embodiment, fluid provided with the fiber implant may contain a combination of the added components.

In yet another method, the filamentous fibers of the present invention are used to treat herniated discs in combination with one or more additional procedures. Illustratively, the filamentous fibers of the present invention are ideally suited for use in conjunction with disc nucleoplasty. The disc nucleoplasty procedure uses a minimally-invasive catheter to remove tissue using radio waves that break up the tissue. The filamentous fibers would then be inserted into the intravertebral disc space, to ease pain and help provide mechanical stability to the damaged disc. Both the nucleoplasty and the insertion of the filamentous fibers may be done through the same very small incision. It is understood, however, that the fibers of the present invention may be used in combination with other procedures.

Although the invention has been described in detail with reference to preferred embodiments, variations and modifications exist within the scope and spirit of the invention as described and defined in the following claims.

1. A method for treating a damaged vertebral disc comprising the steps of
providing a filamentous fiber configured to absorb fluid, and
inserting the filamentous fiber into the damaged disc such that the filamentous fiber absorbs fluid, expands, and provides cushioning to the damaged disc.

2. The method of claim 1, wherein the inserting step includes inserting the filamentous fiber into a disc space through a cannulating needle.

3. The method of claim 2, wherein the inserting step includes using a delivery unit to move the filamentous fiber through the cannulating needle.

4. The method of claim 1, wherein the filamentous fiber is dehydrated.

5. The method of claim 1, wherein the filamentous fiber is a hydrogel.

6. The method of claim 1, wherein the filamentous fiber comprises collagen.

7. The method of claim 1, wherein the vertebral disc is a degenerative disc.

8. The method of claim 7, wherein the degenerative disc has normal disc height.

9. The method of claim 1, wherein the vertebral disc is herniated and further comprising the step of
performing a disc nucleoplasty to remove the herniated portion of the vertebral disc.

10. The method of claim 9, wherein the disc nucleoplasty is performed through a small incision, and the inserting the filamentous fiber step is performed using the same small incision.

11. The method of claim 1, further comprising the step of injecting fluid into the damaged disc.

12. The method of claim 1, further comprising the step of cutting the thread to size prior to or during insertion.

13. The method of claim 1, further comprising the step of inserting a plurality of additional filamentous fibers into the disc.

14. The method of claim 1, further comprising the step of freezing the filamentous fiber prior to insertion.

15. An implant for use in treating a damaged disc comprising a filamentous fiber having potential to absorb fluid and packaged for insertion into disc space.

16. The implant of claim 15, wherein the filamentous fiber comprises a hydrogel material.

17. The implant of claim 15, wherein the filamentous fiber comprises collagen.

18. The implant of any of claims 15-17 wherein the filamentous fiber is dehydrated.

19. A kit for use in treating a damaged disc comprising a dehydrated filamentous fiber packaged for insertion into a disc.

20. The kit of claim 19, wherein the dehydrated filamentous fiber comprises a hydrogel material.

21. The kit of claim 19, wherein the dehydrated filamentous fiber comprises collagen.

22. The kit of claim 19, wherein the filamentous fiber is packaged on a spool.

23. The kit of claim 19, further comprising a delivery instrument.

24. The kit of claim 23, wherein the delivery instrument includes a cannulating needle.

25. The kit of claim 24, wherein the delivery instrument further comprises a delivery unit configured for insertion through the cannulating needle.

26. The kit of claim 25, wherein the filamentous fiber is provided preloaded onto the delivery unit.

27. The kit of claim 19, further comprising a plurality of additional dehydrated filamentous fibers.
